SEQUENCE LISTING (5 sequences)

SEQ ID NO: 1 - protein, 721 aa, Homo sapiens
  misc_feature: amino acid sequence of receptor  ACE2 1-732 (NCBI Reference Sequence NP_001358344.1)
MSSSSWLLLSLVAVTAAQSTIEEQAKTFLDKFNHEAEDLFYQSSLASWNYNTNITEENVQNMNAGDKWSAFLKEQSTLAQMYPLQEIQNLTVKLQLQALQQNGSSVLSEDKSKRLNTILNTMSTISTGKVCNPDNPQECLLLEPGLNEIMANSLDYNERLWAWESWRSEVGKQLRPLYEEYVVLKNMARANHYEDYGDYWRGDYEVNGVDGYDYSRGQLIEDVEHTFEEIKPLYEHLHAYVRAKLMNYPSYISPIGCLPAHLLGDMWGRFWTNLYSLTVPFGQKPNIDVTDAMVDQAWDAQRIFKEAEFFVSVGLPNMTQGFWENSMLTDPGNVQKAVCHPTAWDLGKGDFRILMCTKVTMDDFLTAHEMGHIQYDMAYAAQPFLLRNGANEGFHEAVGEIMSLSAATPKHLKSIGLLSPDFQEDNETENFLLKQALTIVGTLPFTYMLEKWRWMVFKGEIPKDQWMKKWWEMKREIVGVVEPVPHDEYCDPASLFHVSNDYSFIRYYTRTLYQFQFQEALCQAAKHEGPLHKCDISNSTEAGQKLFNMLRGKSEPWTLALENVVGAKNMNVRPLLNYFEPLFTWLKDQNKNSFVGWSTDWSPYADQSIKVRSLKSALGDKAYEWNDNEMYLFRSSVAYAMRQYFLKVKNQMILFGEEDVRVANLKPRISFNFFTAPKNVSDIIPRTEVEKAIRMSRSRINDAFRLNDNSLEFLGIQPTLG

SEQ ID NO: 2 - protein, 5 aa, Artificial Sequence
  Amino acid sequence of the recognized motif by the enzyme Sortase
  misc_feature (3)..(3): Xaa can be any naturally occurring amino acid
LPXTG

SEQ ID NO: 3 - protein, 118 aa, Artificial Sequence
  Amino acid sequence of the Ty1 nanobody (Hanke L. et al. “An alpaca nanobody neutralizes SARS-CoV-2 by blocking receptor interaction” Nat. Comm. 11, N. 4420 (2020))
QVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSS

SEQ ID NO: 4 - protein, 134 aa, Artificial Sequence
  Amino acid sequence of the His-tagged nanobody H11-H4 (Huo J. et al. “Neutralizing nanobodies bind SARS-CoV-2 spike RBD and block interaction with ACE2” Nat. Structural & Molec. Biol., 27, 846-854 (2020))
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSSKHHHHHH

SEQ ID NO: 5 - protein, 134 aa, Artificial Sequence
  Amino acid sequence of the His-tagged nanobody H11-D4 (Huo J. et al. “Neutralizing nanobodies bind SARS-CoV-2 spike RBD and block interaction with ACE2” Nat. Structural & Molec. Biol., 27, 846-854 (2020))
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSKHHHHHH

(19) United States
(12) Patent Application Publication
(10) Pub. No.: US 2024/0310320 A1
(43) Pub. Date: Sep. 19, 2024

(54) FIELD EFFECT TRANSISTOR (FET) BIOSENSOR FOR DETECTION OF VIRAL PARTICLES

(71) Applicant: UNIVERSITA POLITECNICA DELLE MARCHE, Ancona (IT)

(72) Inventors: Daniele DI MARINO, Ancona (IT); Mattia D'AGOSTINO, Ancona (IT); Chiara ARDICCIONI, Ancona (IT); Alice ROMAGNOLI, Ancona (IT); Ilda D'ANNESSA, Ancona (IT); Davide MENCARELLI, Ancona (IT); Eleonora PAVONI, Ancona (IT); Paolo CRIPPA, Ancona (IT); Giorgio BIAGETTI, Ancona (IT); Luca PIERANTONI, Ancona (IT); Marco FARINA, Ancona (IT)

(73) Assignee: UNIVERSITA POLITECNICA DELLE MARCHE, Ancona (IT)

(21) Appl. No.: 18/260,882

(22) PCT Filed: Jan. 12, 2022
(86) PCT No.: PCT/IB2022/050205

§ 371 Date: Jul. 10, 2023

(30) Foreign Application Priority Data
Jan. 13, 2021 (IT) 102021000000533

(51) Int. Cl. G01N 27/414; G01N 33/543; G01N 33/569

(52) U.S. Cl. CPC G01N 27/4145; G01N 33/5438; G01N 33/56983

(57) ABSTRACT

The present invention relates to a field-effect transistor (FET) biosensor and to a device including the biosensor for the detection of viral particles and/or fragments thereof. The invention further relates to the in vitro use, a method and a kit including the biosensor and/or device for the diagnosis of viral infections, as well as a process for the preparation of a field-effect transistor biosensor for the detection of viral particles and/or fragments thereof.

The present invention relates to a field-effect transistor (FET) biosensor and a device comprising it for the detection of entire viral particles and/or fragments thereof. The invention further relates to the in vitro use, a method, and a kit comprising said biosensor and/or device for the diagnosis of viral infections, as well as a process for the preparation of a field-effect transistor biosensor for the detection of viral particles and/or fragments thereof.

### STATE OF ART

The infections of viral nature, and in particular the acute viral respiratory infections, represent the main infectious cause of death in the world. The laboratory diagnosis of a viral infection generally provides for the identification of virus RNA by applying techniques based upon the polymerase chain reaction (PCR) and immunization methods, such as ELISA, for the detection of antibodies against the virus in serum samples obtained from a patient.

These methods are based upon a detection of indirect type of virus and result to be a little effective in detecting the infectious cases: the presence of viral RNA does not provide information about the subject's infectivity, whereas the presence of antibodies in the serum does not involve that the infections is still in progress. Moreover, these methods are not quick: the antibodies, for example, can be detected only after several days as from onset of symptoms, whereas the PCR method requires several hours to obtain the result and requires specialized personnel and machines.

Therefore, such methods are not capable of meeting the needs of quick and early diagnosis of the viral infection or of clinical screening in the initial phases of the infection.

In particular, in case of SARS-COV-2 infections, the detection of viral RNA in a test with swab does not represent a sufficient condition so that the infectivity of the subject under consideration can be demonstrated.

Among the various diagnostic tools currently available on the market, the electric biosensors based upon field-effect transistors (FET) offer several advantages for a quick, “label-free”, and selective detection of viral particles.

In this field, the field-effect transistors based upon graphene as channel for the charge transportation, in particular, represent very promising alternatives to the metal-oxide-semiconductor field-effect transistors (MosFET). The graphene consists of a monolayer of carbon atoms arranged to form a hexagonal lattice, characterized by a linear energy dispersion up to 1 eV and by an almost symmetric behaviour for lacunae and electrons, described as zero-mass Dirac fermions. The first quick graphene-based and broadband device dates back to 2010 (IBM), with a bandwidth equal to 100 GHz, and several developments followed it. The graphene shows excellent properties, such as the capability of supporting high currents (>1 mA/μm), high mobility (>100,000 cm²V⁻¹s⁻¹), a reduced dimensionality from the chemical point of view and, then, the potential capability of minimizing possible “short-channel” effects. Besides, possible limitations linked to the use of graphene derive from its ambipolarity and from the absence of bandgap.

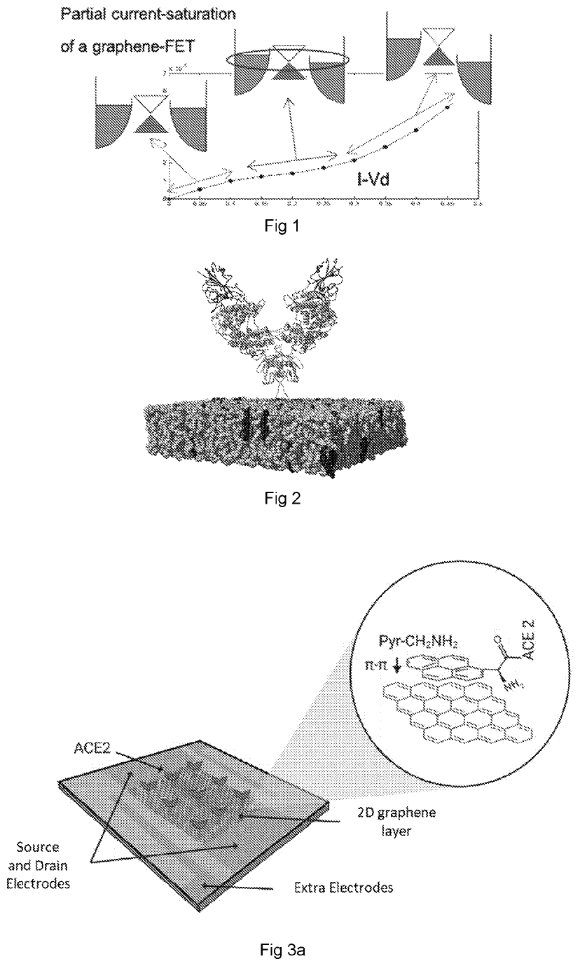

A numerical characterization of the current-voltage curves (curves I-V) in the graphene shows a cancellation of density of the energy states at Dirac point, a phenomenon which partially compensates the absence of bandgap. Such phenomenon can involve a sub-optimum behaviour (FIG. 1) in terms of difficulties to reach the current saturation (where a low saturation involves a reduced gain), and it can minimize the “sub-threshold” slope. However, the high mobility and conformability to a technology of planar type still represent important advantages in favour of the implementation of graphene-based FETs in applications in biosensor field.

As in a FET system the current is modulated by means an external gate voltage VG, the use of a material with high dielectric constant, for example a gate insulator, should allow an optimization of the transistor performances. Clearly, the substrate can degrade the graphene electronic properties, then a careful selection of the materials and of the manufacturing technique is required (for example, it is known that the mobility of the graphene transferred on a SiO₂/Si substrate is limited to 10000 cm²V⁻¹s⁻¹).

The biosensors represent interesting examples of graphene-FET based devices, since in such systems a modulation of the graphene surface charge is exploited to detect the molecular interactions and/or the binding with proteins or other biomolecules of interest.

One of the problems which has been always found in various biotechnological applications, including biosensors, is the absence up to now of a system allowing to orient the biological macromolecules on the surface of graphene, or other materials, in controlled manner. This represents an important experimental limit, since the interactions between proteins involve specifically only some well precise portions of the proteins themselves.

In this context, then, the need is still much felt for having available biosensors for the detection of viral particles characterized indeed by greater rapidity and selectivity, but at the same time by a high sensitivity in the detection.

### SUMMARY OF THE INVENTION

The herein proposed invention relates to a new biosensor characterized by high specificity and sensitivity for use in the detection of viral particles and/or fragments thereof. In particular, the authors of the present invention have devised an effective strategy for the functionalization of a field-effect transistor (FET) biosensor, comprising the immobilization on the active area of the sensor of a protein, for example a viral receptor or a nanobody (Nb), capable of specifically binding a viral particle and/or a fragment thereof. This strategy of site-specific conjugation allows to orient in a controlled way said protein on the surface of the biosensor substrate; the binding between the molecules precisely oriented with the viral particles is capable of generating a variation in the substrate charge mobility, which is capable of determining a change in the electric conductivity allowing to transduce the biological recognition event in a detectable electric signal.

Surprisingly, this translates into a high sensitivity and specificity in the detection of viral particles or fragments thereof. Apart from having optimum sensitivity and selectivity, the so developed biosensor is characterized by a high ease of use, as well as by a considerable response rapidity in the detection of viral particles, ideal features for a timely diagnosis of infections.

According to an aspect of the invention, said biosensor consists of a graphene-based FET a functionalized with viral receptors suitable oriented for the specific binding with viral particles. The optimum conductivity and high specific surface area of graphene makes this material particularly effective for the development of applications in biological and electrochemical field.

In particular, an embodiment according to the invention relates to a graphene-based FET (gFET) biosensor functionalized with engineered bio-molecules of human angiotensin conversion enzyme 2 (ACE2), or with nanobodies, suitably oriented on the graphene surface so as to bind specifically SARS-COV-2 virus. In this preferred configuration, ACE2 enzyme is suitably oriented by means of a bio-conjugation reaction based upon hybridization between two probes of complementary DNA.

Advantageously, the biosensor capability of detecting whole viral particles can be exploited both for the detection of the virus existing in the environment, for example in air or water samples, and for the quick diagnosis of infections of viral nature, for example SARS-COV-2 infections, by easing the isolation of the most infectious subjects, and by allowing equally to obtain important information related to the pathology prognosis. Such capability is confirmed by the results in the experimental tests performed by the authors of the present invention (Example 6), which show that the biosensor, the invention relates to, results to be capable of detecting, with surprising specificity, the presence of each variant of SARS-COV-2 virus known up to now in biological samples obtained from real patients.

As shown in the detailed description, the biosensor the present invention relates to can be easily integrated within a portable device and thus it can be actuated even in home environment by not experienced personnel.

Therefore, the present invention relates to:
a field-effect transistor (FET) biosensor for the detection of viral particles and/or fragments thereof comprising a substrate and a protein capable of specifically binding a viral particle and/or a fragment thereof, immobilized on the surface of said substrate, wherein said protein is immobilized so that the binding of a viral particle and/or a fragment thereof to said protein determines the emission of a detectable electrical signal;
a portable device comprising at least a field-effect transistor biosensor according to any one of the herein described embodiments;
the in vitro use of a biosensor or a device according to any one of the herein described embodiments for the diagnosis of viral infections and the detection of viral particles in the environment;
a method for detecting the presence of a viral particle and/or a fragment thereof within a biological sample and/or for the diagnosis of a viral infection comprising the following steps:
   i. contacting a biological sample to be analysed with a biosensor as defined and/or with a device comprising one or more biosensors as described herein; and
   ii. detecting binding reactions with a viral particle and/or a fragment thereof by means of an electrical signal emitted by said one or more biosensors.
a process for the preparation of a field-effect transistor (FET) biosensor for the detection of viral particles and/or fragments thereof comprising the following steps:
   i. arranging a FET biosensor comprising at least a substrate;
   ii. functionalizing said at least a substrate with a linker molecule capable of binding a first single-stranded oligonucleotide;
   iii. binding a protein capable of specifically binding a viral particle and/or a fragment thereof to a second single-stranded oligonucleotide comprising a nucleotide sequence complementary to the nucleotide sequence of said first oligonucleotide;
   iv. conjugating said receptor bound to said second oligonucleotide to said substrate, by hybridization of said first oligonucleotide with said second oligonucleotide;
a kit for the detection of a viral particle and/or a fragment thereof and/or for the diagnosis of a viral infection comprising a biosensor and/or a device according to any one of the herein described embodiments, and one or more control reagents.

Additional advantages, as well as the features and use modes of the present invention will result evident from the following detailed description.

### DETAILED DESCRIPTION OF FIGURES

FIG. 1. Typical I-VD curve of graphene-FET, wherein a quasi-saturation of current is shown (central portion of the curve).

FIG. 2. Atomic model of ACE2 receptor immersed in a phospholipidic double layer containing also molecules of cholesterol, interacting with two RBD domini used to perform simulations of molecular dynamics.

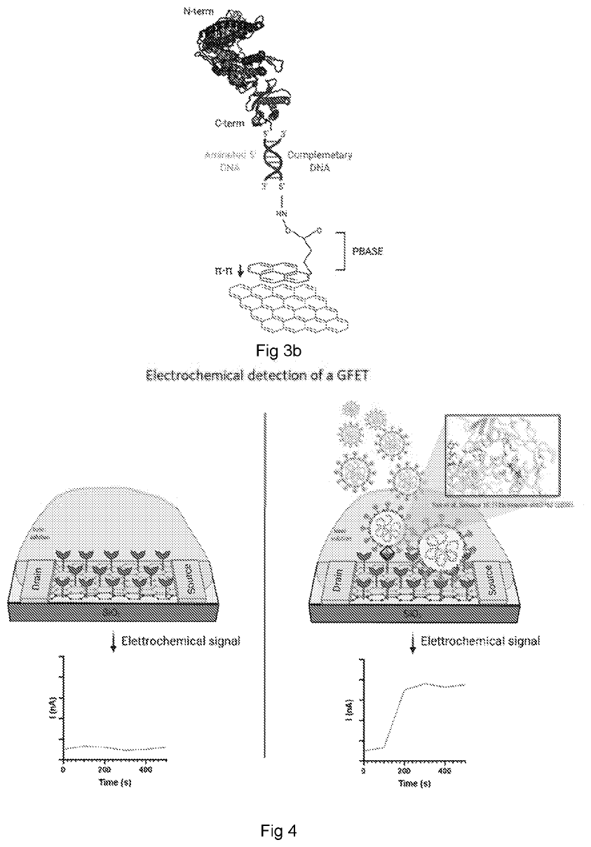

FIG. 3. Graphic representation of an embodiment of a biosensor according to the invention. (a) Graphene-based field-effect transistor (gFET) functionalized with recombinant ACE2. (b) New functionalization strategy: the C-terminal group of ACE2 is bound to 5′-end of a small DNA oligo (5′-GCACTG-3′). This ACE2-DNA Chimera will be hybridized with a complementary DNA probe modified with an amine group (H2N-5′-CAGTGC-3′), previously bound to the graphene surface by means of PBASE.

FIG. 4. Schematic representation of the electric approach applied to the reading of signal coming from the gFET biosensor. The graphs represent only a hypothetical output signal coming from the device.

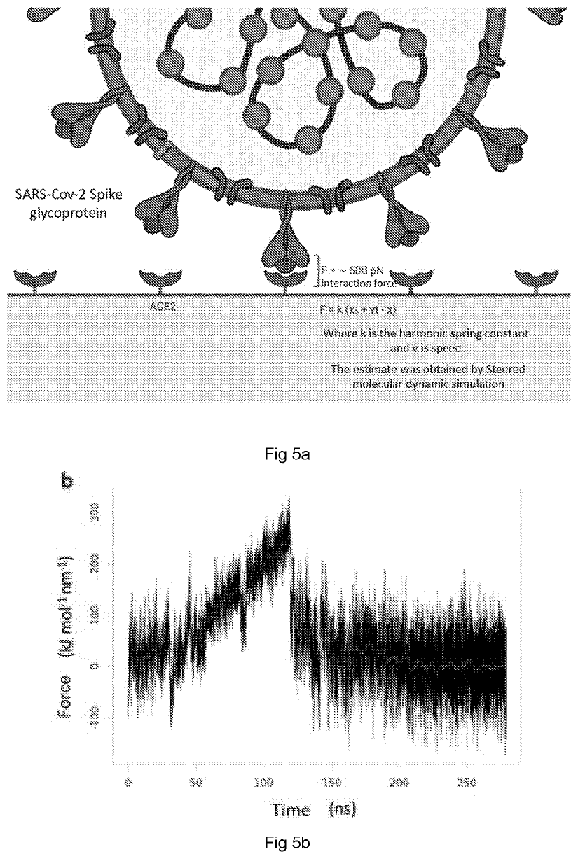

FIG. 5. (a) Schematic view of the interaction between the ACE2 peptidase domain and SARS-CoV-2 Spike protein. (b) Graph of the interaction force obtained by means of simulation of oriented molecular dynamics (SMD). The force spectrum represents the detachment process between ACE2 and RBD during 275 ns of simulation with a traction speed v=0.05 Å/ns.

FIG. 6. Atomic force microscopy (AFM) images of (a) graphene, (b) graphene modified with PBASE, (c) graphene functionalized with ACE2 incubated with Spike protein (S1) of SARS-COV-2. The bottom graphs show the height profiles in the three corresponding AFM images. The scale is equal to 10 nm² for figures (a) and (c) and 3 nm² for figure (b).

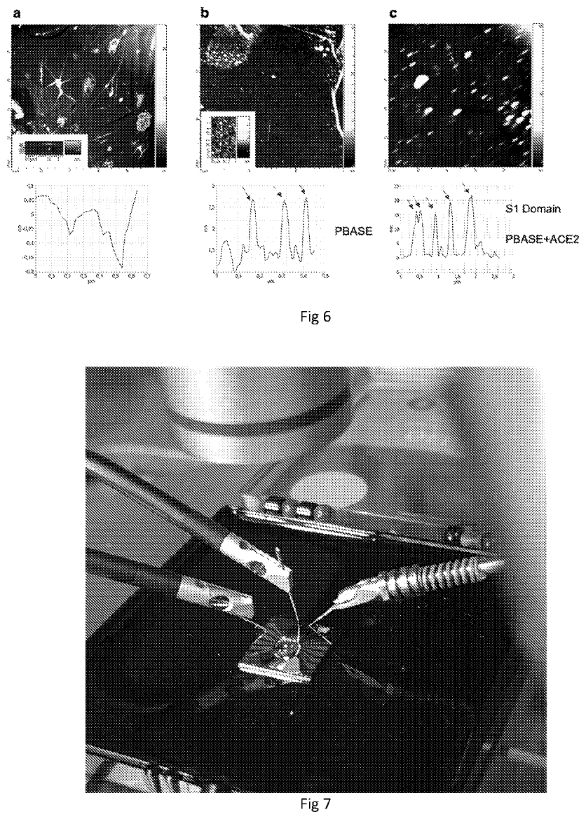

FIG. 7. Series of graphene-FETs functionalized during a measurement.

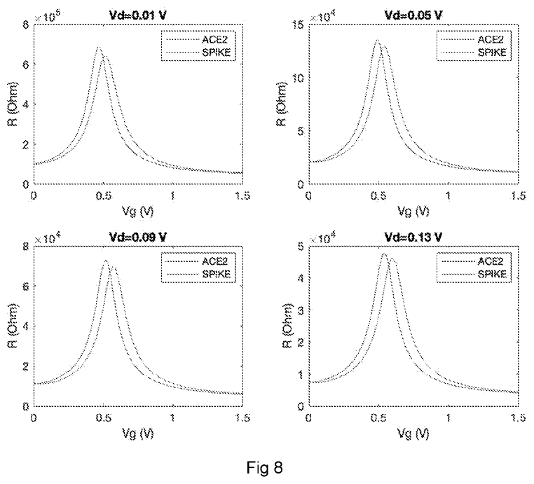

FIG. 8. Channel resistance for four drain-source voltages (10, 50, 90 and 130 mV) in function of the gate-source voltage, comprised between 0 and 1.5 V. The resistance is shown both the functionalization of liquid gate with ACE2 receptor (blue line) and with SARS-COV-2 Spike protein (red line), respectively.

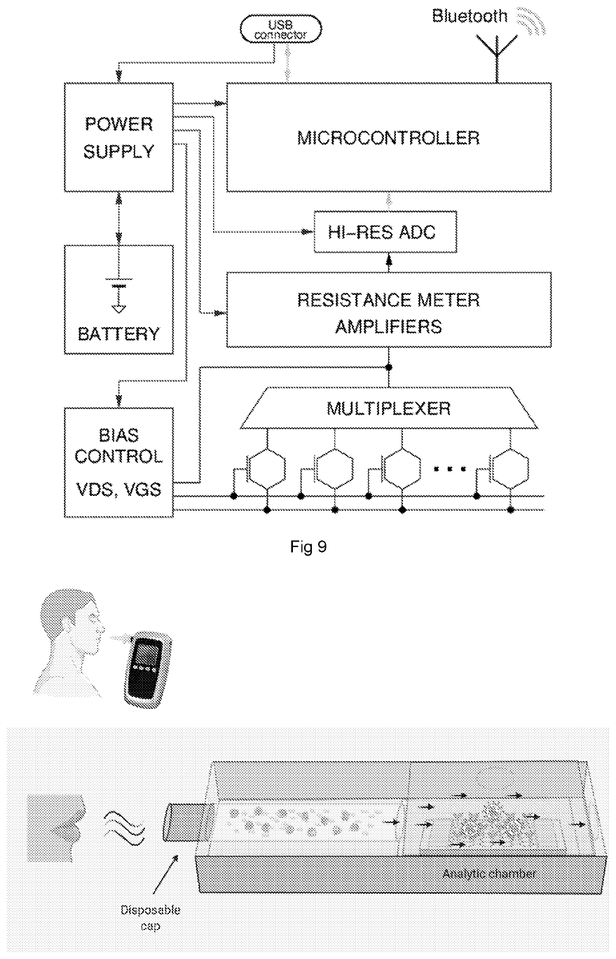

FIG. 9. Block diagram of an embodiment of the prototype of the device according to the invention.

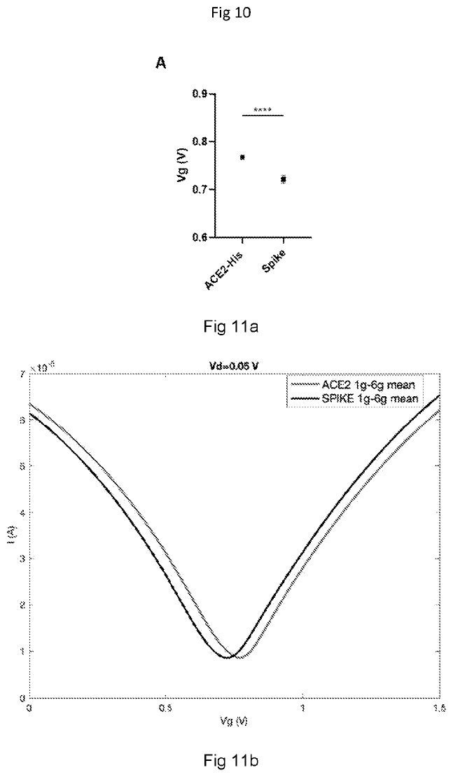

FIG. 10. Representation of a possible application of the biosensor according to the present invention (“breathalyser”).

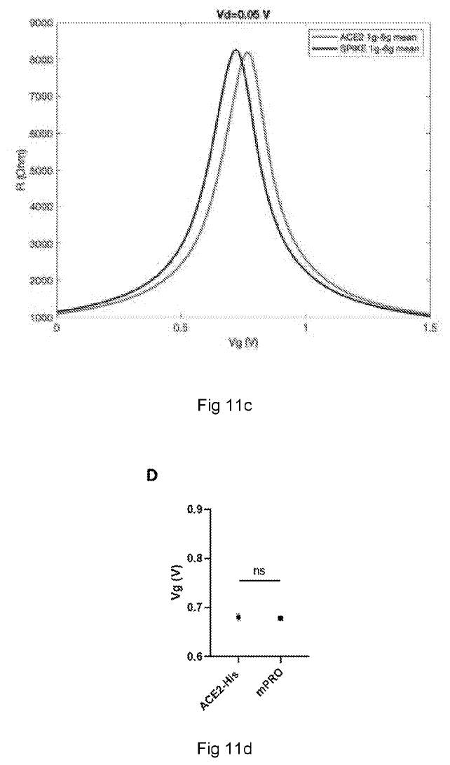

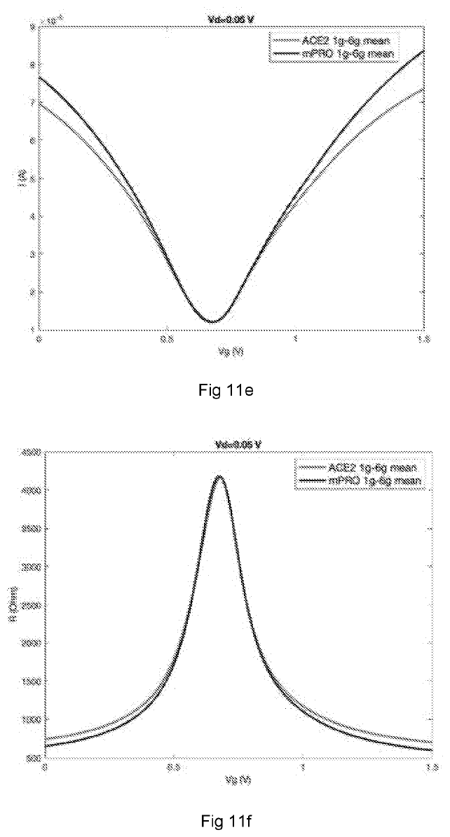

FIG. 11. (A and D) Difference in gate voltage (V) of gFET conjugated with ACE2-His before and after incubation with 2 μg/mL of SARS-COV-2 trimeric Spike protein (A) or SARS-COV-2 mPRO protein (D), respectively; (B and E) I(A)-Vg(V) output curves of FET conjugated with ACE2-His before (light line) and after (dark line) the addition of 2 μg/mL of trimeric Spike protein (B) or of mPRO protein (E), respectively; (C and F) RΩ(D)-Vg(G) output curves of FET conjugated with ACE2-His before (light line) and after (dark line) the addition of 2 μg/mL of trimeric Spike protein (C) or of mPRO protein (F), n=6, (t Welch test).

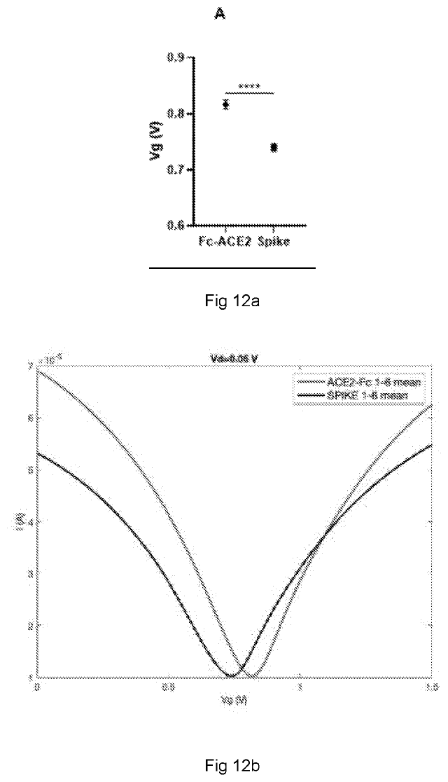

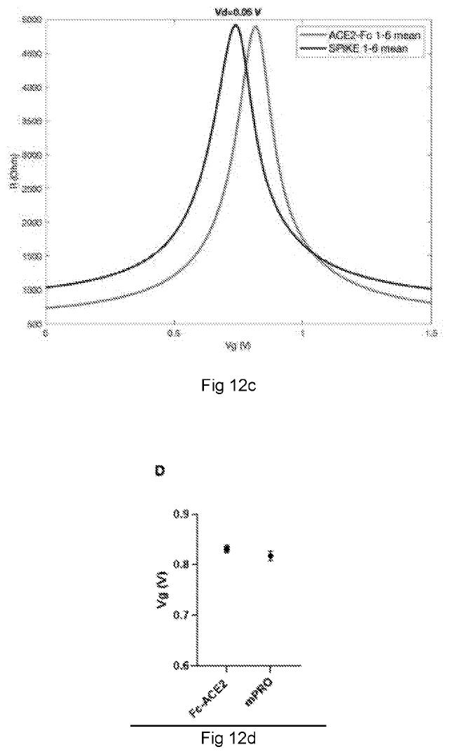

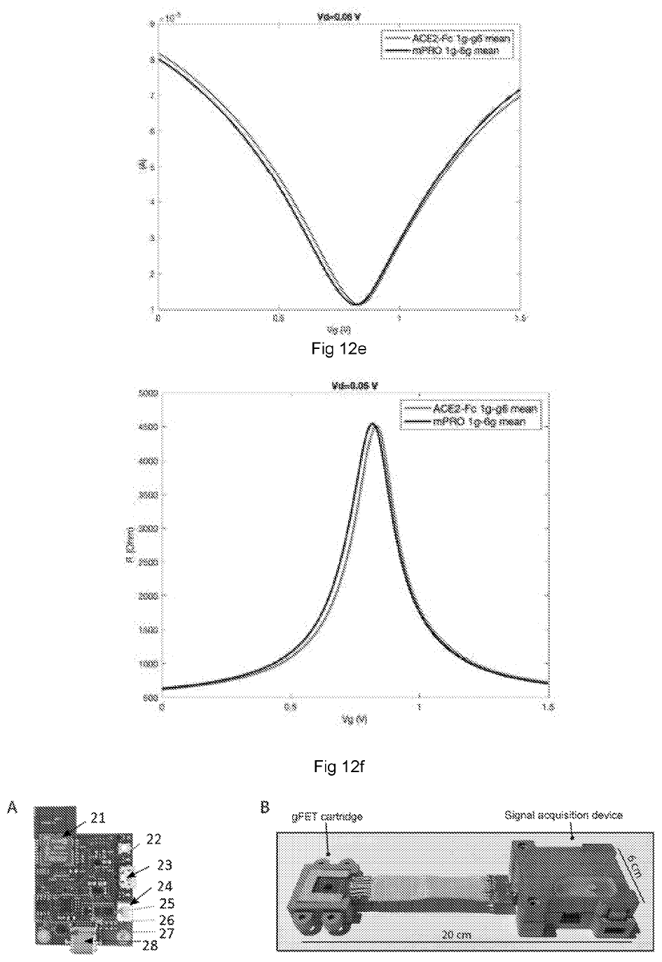

FIG. 12. (A and D) Difference in gate voltage (V) of gFET conjugated with ACE2-Fc before and after incubation with 2 μg/mL of SARS-COV-2 trimeric Spike protein (A) or SARS-COV-2 mPRO protein (D), respectively; (B and E) I(A)-Vg(V) output curves of FET conjugated with ACE2-Fc before (light line) and after (dark line) the addition of 2 μg/mL of trimeric Spike protein (B) or of mPRO protein (E), respectively; (C and F) RΩ(D)-Vg(G) output curves of FET conjugated with ACE2-Fc before (light line) and after (dark line) the addition of 2 μg/mL of trimeric Spike protein (C) or of mPRO protein (F), n=6, (t Welch test).

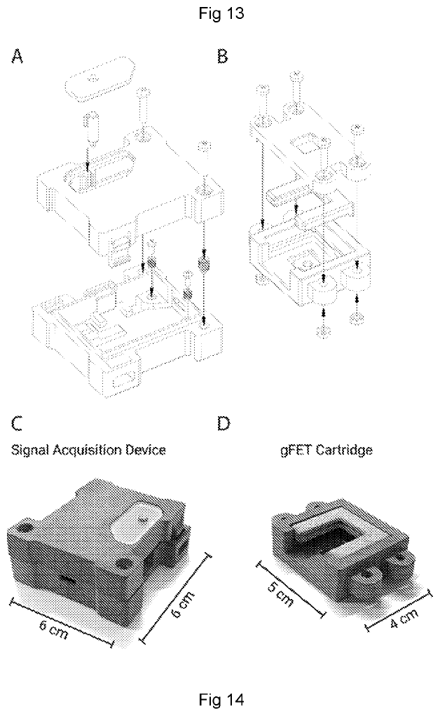

FIG. 13. (A) Device electronic module. The designated elements correspond to the following ones: (21) microcontroller and BLE radio; (22) power supply and management element; (23) charging connector; (24) battery connector; (25) MUX; (26) ADC; (27) DAC; (28) connector of gFET cartridge. (B) Photo of the whole POC prototype.

FIG. 14. (A) Casing assembly: window, button, upper casing, lower casing (from top to bottom); (B) Assembly of the complete Carrier: Carrier 3, Carrier 1 and Carrier 2 (from top to bottom). Photos of the device units: main casing (C) and casing for gFET (D).

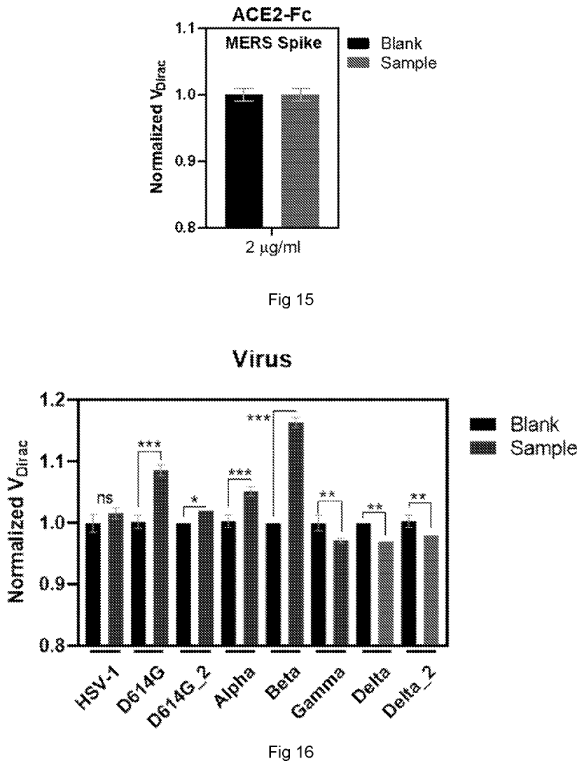

FIG. 15. Comparative bar graph of the obtained signal, with chip functionalized with ACE2-Fc, before (in black) and after (in grey) incubation with 2 μg/ml of MERS-COV recombinant Spike.

FIG. 16. Comparative bar graph of the obtained signal, with chip functionalized with ACE2-Fc, before (in black) and after (in grey) incubation of different solutions containing depowered virus of Herpes Simplex Virus-1 (used as negative control); SARS-COV-2 variants D614G, Alpha, Beta, Gamma, Delta.

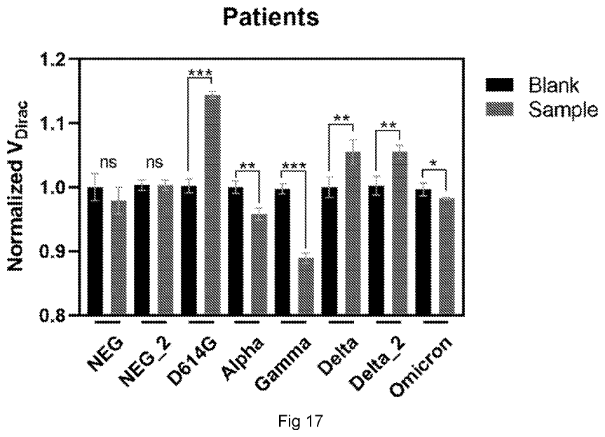

FIG. 17. Comparative bar graph of the obtained signal, with chip functionalized with ACE2-Fc, before (in black) and after (in red) incubation of different samples of patients preserved in PBS 1×.

### GLOSSARY

The terms used in the present description are as generally understood by the person skilled in the art, except where differently designated.

The term “Spike protein” used in the context of the present description relates to the transmembrane trimeric glycoprotein (also known as glycoprotein S) existing in the most external layer of SARS-COV-2 virus, whose monomers consists each one by two subunits (S1 and S2). The sequence of such protein is available in data bank as surface glycoprotein Severe acute respiratory syndrome coronavirus 2 QHD43416.1

It is well known in literature that the interaction between SARS-COV-2 virus and ACE2 enzyme, by recognition of glycosylated Spike viral protein (S) on virion surface, favours the entrance of viral particles within the epithelial cells of the human respiratory apparatus, by triggering the infective process. The resolved molecular structures of Spike proteins suggest the presence of one single monomer trapped with the receptor-binding domain (RBD) in the active configuration (up), required to bind ACE2 and to trigger the invasion of the host cells.

Under the expression “active area, surface or region of the sensor”, in the context of the present description, the region of the biosensor substrate is meant, whereon the recognition reaction or biological binding takes place, which translates into a variation in the charge mobility of the substrate, and then a change in the electric conductivity of the same.

Under the expression “viral receptor” a protein is meant wherein at least a portion thereof is expressed on the cell surface and it is capable of binding at least a virus, in particular one or more proteins of the viral particle.

Under the term “nanobody” or “nanobodies” (in short “Nb” or “Nbs”, also known as “single-domain antibodies”) V_{H}H (variable heavy chain) antibodies are meant, that is minimum monomeric domains for the binding with antigen, derived from single-chain antibodies of camelids (dromedary, llama, camel, alpaca, and so on). Notwithstanding they are about one tenth of the sizes of a conventional antibody (˜ 15 kDa), they keep a specificity and affinity similar to the conventional antibodies, but they are much easier to be cloned, expressed and manipulated, since they are expressed in the bacterial systems in great amounts and at low cost.

The nanobodies have the same structural architecture of V_{H} domains of human immunoglobulins: four regions with preserved sequence (FR1/2/3/4) surrounding three hypervariable rings involved in the binding with antigen (CDR 1/2/3).

Nbs have been developed capable of neutralizing both Spike and RBD of SARS-COV-2.

Under the term “protein capable of specifically binding a viral particle and/or a fragment thereof” a protein is meant which has a specific affinity for a virus portion, for example it is capable of binding a viral protein with a dissociation constant lower than 10 mM, preferably lower than 100 nM, measured for example by means of an in-vitro binding assay.

### DETAILED DESCRIPTION OF THE INVENTION

In a first aspect the invention relates to a field-effect transistor (FET) biosensor for the detection of viral particles and/or fragments thereof comprising at least a substrate and a protein capable of specifically binding a viral particle and/or a fragment thereof, immobilized on the surface of said substrate, wherein said protein is oriented/immobilized so that the binding of a viral particle and/or a fragment thereof with said immobilized protein determines the emission of a detectable electrical signal.

As clearly demonstrated in the experimental section of the present description, the functionalization of the surface of said substrate, which represents the active area or region of the biosensor, with said protein capable of specifically binding a viral particle, suitably oriented, allows to obtain not only a high selectivity in recognizing the viral particles, but above all a high sensitivity in the detection. The electric signal generated at the surface of the substrate in response to the binding reaction, then recognition reaction, of the immobilized protein with the viral protein, can be amplified and analysed, then correlated to the presence of virus within a sample subjected to analysis.

According to an aspect of the invention, said protein then is a protein capable of binding in a specific and selective way a viral particle and/or a fragment thereof.

Under “fragment” in the context of the present description any portion of the viral particle capable of specifically binding said protein is meant, for example a polypeptide including at least a sufficient portion to confer to said polypeptide a specific binding to said protein.

In an embodiment according to the invention, said protein in particular is a protein capable of binding a viral particle of SARS-COV-2 and/or a fragment thereof.

According to an aspect of the present invention, said protein is a protein binding specifically to a region of Spike protein (S) of SARS-COV-2 virus. In an embodiment, said region is i) in the S1 domain of SARS-COV-2 S protein; or (ii) in SARS-COV-2 S protein trimer in its pre-melting shape thereof; or (iii) in SARS-COV-2 S protein in its post-melting shape; or (iv) in the receptor-binding domain (RBD) of SARS-COV-2 S protein or in a combination thereof.

According to an aspect of the present invention, said protein capable of specifically binding a viral particle and/or a fragment thereof according to any one of the previously described variants is a viral receptor or un nanobody.

In an embodiment of the present invention, said viral receptor in particular is a receptor capable of specifically binding a viral particle of SARS-COV-2 and/or a fragment thereof, in particular it is a human receptor.

Viral receptors suitable to be used for the functionalization of a biosensor according to the present invention are recombinant viral receptors, that is produced in recombinant way within host cells by using standard methods known in the art.

According to an aspect of the present invention, said receptor is the angiotensin-converting enzyme 2, also known with the acronym ACE2, and/or a fragment thereof, in particular it is the human ACE2 enzyme. In an embodiment according to the present invention, said receptor is the ACE2 enzyme having the aminoacidic sequence SEQ ID Nr. 1 and/or a fragment thereof.

Said ACE2 receptor consists of a collectrin-like C-terminal domain ending with a single transmembrane helix and of a peptidase N-terminal domain which is responsible for the interaction with RDB (FIG. 2).

Said ACE2 receptor could be a recombinant ACE2 receptor.

According to an aspect of the present invention, said ACE2 receptor can include a peptide tag at the C-terminal end of its aminoacidic sequence. Examples of protein tags include a polyhistidine tag (His-tag) or Fc tag (Fc-tag). In the latter case, the ACE2 receptor comprising said Fc tag is also known as Fc-ACE2 Chimera or Fc-ACE2 melting protein.

His-tag is a protein tag widely used in order to purify recombinant proteins, and consists of an aminoacidic chain with a length from 6 to 14 units of histidine joined by peptide bonds. Fc-tag derives from Fc domain of an immunoglobulin (Ig).

Preferably, said receptor is the ACE2 enzyme having sequence SEQ ID Nr. 1 and/or a fragment thereof, wherein one or more of the following mutations are inserted in the sequence of said enzyme: T27Y, L79T, N330Y, preferably all three mutations are inserted. Advantageously, the latter variant of ACE2 enzyme is characterized by a high binding affinity for SARS-COV-2 Spike protein.

Preferably, said receptor is human ACE2 enzyme engineered as described in the publication of K. K. Chan et al. “Engineering human ACE2 to optimize binding to the spike protein of SARS coronavirus 2”, Science Vol 369, Issue 6508, pages 1261-1265 (2020), herein integrally incorporated by reference.

In an embodiment of the present invention, said receptor is the ACE2 enzyme and/or a fragment thereof according to any one of the previously described variants, comprising LPxTG (Leu-Pro-X-Thr-Gly) motif, SEQ ID Nr. 2, at C terminal end of its aminoacidic sequence, wherein X can be any amino acid.

A nanobody which can be used for implementing a biosensor according to any one of the herein described variants in particular is a V_{H}H antibody derived from single-chain antibodies of camelids, capable of specifically binding a viral particle of SARS-COV-2 and/or a fragment thereof.

Not limiting examples of nanobodies which can be used for implementing a biosensor according to the present invention are nanobodies as described in the following scientific publications herein integrally incorporated by reference: Hanke L. et al. “An alpaca nanobody neutralizes SARS-COV-2 by blocking receptor interaction” Nat. Comm. 11, N. 4420 (2020); Huo J. et al. “Neutralizing nanobodies bind SARS-COV-2 spike RBD and block interaction with ACE2” Nat. Structural & Molec. Biol., 27, 846-854 (2020); Schoof M. et al. “An ultrapotent synthetic nanobody neutralizes SARS-CoV-2 by stabilizing inactive Spike”, Science, Vol. 370, Issue 6523, pages 1473-1479 (2020); Xiang Y. et al. “Versatile and multivalent nanobodies efficiently neutralize SARS-COV-2”, Science, Vol. 370, Issue 6523, pages 1479-1484 (2020).

In a preferred embodiment of the present invention, said nanobody is a nanobody capable of binding the receptor binding domain (RBD) of SARS-COV-2 S protein, in particular it is a nanobody having an aminoacidic sequence selected among SEQ ID Nr 3, SEQ ID Nr 4, SEQ ID Nr 5.

An aspect of the present invention relates to a FET biosensor as previously described, wherein said at least a substrate is a graphene substrate.

The binding between said protein molecules precisely oriented with the viral particles generates a variation in the charge mobility of graphene capable of determining a change in the electric conductivity; in this way the biological recognition event is translated into a detectable electric signal.

According to an aspect of the present invention, said protein is immobilized on the surface of said substrate through the binding of a group of said protein with a linker molecule conjugated to the surface of said substrate.

In other terms, said protein is immobilized on the surface of said substrate by covalent binding between a functional group of said protein and a functional group of a linker molecule previously conjugated on the surface of said substrate.

A linker molecule suitable to be used for the binding with said protein, and then for the protein immobilization on substrate, is a bi-functional molecule consisting of a first functional group, at one end of the molecule, which is capable of conjugating said linker molecule to the surface of the substrate, and of a second functional group, at the other end of the molecule itself, capable of binding covalently a functional group existing in said protein. A linker molecule suitable to be used for immobilizing said protein on the surface of said substrate in particular is a linker molecule comprising a functional group capable of binding covalently only a specific portion, or group, of said protein, so as to define univocally the orientation of said protein on the surface of said substrate.

Preferably, according to the present invention, said group of said protein is the C-terminal portion of the aminoacidic sequence of said protein.

According to an aspect of the present invention, said protein is immobilized on the surface of said substrate by hybridization of a first single-stranded oligonucleotide bound to said protein with a second single-stranded oligonucleotide bound to a linker molecule conjugated to the surface of said substrate. In other terms, said first oligonucleotide will include a nucleotide sequence complementary to a nucleotide sequence of said second oligonucleotide, so that the hybridization of said first oligonucleotide with said second oligonucleotide determines the immobilization of the protein on the substrate.

According to an aspect of the present invention, said first single-stranded nucleotide is an oligonucleotide comprising a functional group capable of specifically binding a group of said protein, whereas said second single-stranded oligonucleotide is an oligonucleotide comprising a functional group capable of binding covalently said linker molecule immobilized on the surface of said substrate.

According to an aspect of the invention, the 5′-end of said first oligonucleotide is bound to the C-terminal portion of the aminoacidic sequence of said protein. The binding between said protein and said first oligonucleotide can be obtained by using any technique known in the art for the formation of protein-oligonucleotide (or DNA-protein) hybrid complexes.

According to an aspect of the present invention, said first oligonucleotide is an oligonucleotide modified with oligoglycine, preferably an oligonucleotide modified with Gly-Gly-Gly (Gly₃) motif.

Moreover, in an embodiment according to the present invention, said protein capable of specifically binding a viral particle and/or a fragment thereof is a recombinant protein containing the motif having SEQ ID Nr. 2 at C-terminal end of its aminoacidic sequence.

In an embodiment, said first oligonucleotide is bound to said protein by means of a conjugation reaction mediated by the sortase enzyme, for example according to the protocol described in the publication of M. A. Koussa et al. “Protocol for sortase-mediated construction of DNA-protein hybrids and functional nanostructures”, Methods Vol. 66, Issue 2, pages 134-141 (2014), herein integrally incorporated by reference. Said sortase enzyme (sortase A) in presence of a recombinant protein substrate (in a recombinant ACE2 embodiment), containing a LPxTG motif at C-terminal and an oligonucleotide derivatized with oligoglycine, is capable of exerting a transpeptidation reaction between the protein and the oligoglycine Chimera (e.Gly 3), by forming a peptide bond.

According to an additional aspect of the present invention, said second oligonucleotide is a modified oligonucleotide so as to include a functional group capable of binding covalently a functional group of said linker molecule conjugated on the substrate surface. According to the used linker molecule A, even based upon the type of substrate, the person skilled in the art could select a modified oligonucleotide so as to include the most suitable functional group for the binding with said linker molecule.

In an embodiment according to the present invention, said second oligonucleotide comprises an amino group at the 5′-end capable of binding said linker molecule conjugated to the surface of said substrate.

As previously mentioned, a linker molecule suitable to be used for the conjugation of said second oligonucleotide on the surface of said substrate, and then for the immobilization of said protein on the substrate itself, is a bi-functional linker molecule. In particular, in an embodiment according to the present invention said linker molecule is 1-pyrenebutyric acid N-hydroxy succinimide ester.

1-pyrenebutyric acid N-hydroxy succinimide ester is characterized by a pyrene group, binding to a graphene substrate through an interaction of TT-TT stacking type, and by a portion consisting of a succinimide group extending towards the substrate external surface allowing the immobilization of an oligonucleotide comprising, for example, an amino group at the 5′-end.

According to an aspect of the present invention, said first and second oligonucleotide can be short sequences of single-stranded DNA.

Examples of oligonucleotides suitable to be used for the immobilization of said protein on the surface of said substrate by hybridization, are single-stranded oligonucleotides having a number of bases for example comprised between 6 and 8. Said oligonucleotides have a sufficient length so that a stable hybridization with the complementary oligonucleotide takes place and, in the same way, they make the formation of secondary structures disadvantageous. The use of sufficiently short oligonucleotides further allows not to move away excessively said protein from the substrate surface, a crucial aspect for controlling the biosensor sensitivity.

According to an aspect of the present invention, said first oligonucleotide has the nucleotide sequence 5′-GCACTG-3′ whereas said second oligonucleotide has the sequence H2N-5′-CAGTGC-3′. In an embodiment of the present invention, said first oligonucleotide has the sequence 5′-GCACTG-3′ and said second oligonucleotide has the sequence 5′-CAGTGC-3′, wherein said second oligonucleotide comprises an amino group at the 5′-end.

In an aspect of the present invention, said first oligonucleotide having sequence 5′-GCACTG-3′ is modified with Gly-Gly-Gly motif.

A preferred embodiment of the present invention relates to a FET biosensor for the detection of SARS-COV-2, and in particular for the detection of the whole SARS-COV-2 viral particle, comprising at least a substrate made of graphene and the ACE2 receptor immobilized on the surface of said substrate, wherein said ACE2 receptor is immobilized on the surface of said substrate by hybridization of a first oligonucleotide, bound to the C-terminal portion of said ACE2 receptor, with a second oligonucleotide bound to a molecule of 1-pyrenebutyric acid N-hydroxy succinimide ester (PBASE), which is conjugated to the surface of said substrate by means of x-x stacking. In this particular configuration, ACE2 enzyme, immobilized through the C-terminal end, leaves exposed towards the solvent the area of interaction with the target viral protein, that is the N-terminal portion. The suitably oriented receptor thus is capable of binding in an effective and selective way the Spike protein of the viral particle of SARS-COV-2.

An embodiment according to the invention in particular relates to a FET biosensor for the detection of SARS-COV-2 comprising at least a substrate made of graphene and ACE2 enzyme having SEQ ID N. 1 immobilized on the surface of said substrate, wherein in the sequence of said enzyme one or more of the following mutations are inserted: T27Y, L79T, N330Y, and wherein said ACE2 enzyme is immobilized on the surface of said substrate by hybridization of a first oligonucleotide having sequence 5′-GCACTG-3′, bound to the C-terminal portion of said ACE2 receptor, with a second oligonucleotide having sequence 5′-CAGTGC-3′ bound to a molecule of 1-pyrenebutyric acid N-hydroxy succinimide ester (PBASE), which is conjugated to the surface of said graphene substrate by means of x-x stacking.

As mentioned above, thanks to the generation of a detectable electric signal in response to binding of said viral particles with the properly oriented proteins immobilized on the substrate surface, the FET biosensor according to any one of the herein described embodiments can be used for the detection of viral particles and/or of fragments thereof within a biological sample for diagnostic purposes. According to an embodiment, said viral particles and/or fragments thereof are viral particles and/or fragments of SARS-COV-2 virus.

Therefore, the invention further relates to a biosensor according to any one of the previously described embodiments for use in the diagnosis of viral infections, in particular SARS-COV-2 infections.

According to an aspect of the present invention, a biosensor according to any one of the previously described variants can include a commercially available graphene-based FET (gFET) device. An example of commercially available graphene-based FET which can be used for implementing a biosensor according to the invention is the gFET-S20 graphene-based chip produced by Graphenea.

An aspect of the present invention in particular relates to a biosensor according to any one of the previously described embodiments, comprising at least a graphene-based FET device as described in the gFET-S20 (Graphenea) product data sheet, herein incorporated as reference.

In an embodiment according to the invention, said biosensor comprises a graphene-based chip having dimensions equal to 10 mm×10 mm. Said graphene-based chip can include a plurality of graphene-based field-effect transistors. According to an aspect of the invention, said graphene-based chip in particular consists of 12 graphene-based field-effect transistors.

Advantageously, a biosensor according to any one of the herein described embodiments can be incorporated within a portable device for a quick and effective detection of viral particles existing within a biological sample of a subject.

The present invention then also relates to a portable device comprising at least one field-effect transistor biosensor according to any one of the previously described embodiments.

Since the current technology of production of field-effect transistors, and in particular graphene-based FETs, is not capable of guaranteeing the 100% yields, said device could include an array (or series) of biosensors as above described, so that possible malfunctions of single units could be isolated and that the dispersion in the electrical features of the operating sensors could be mediated. Therefore, according to an aspect of the invention, said device could include a plurality of said biosensors arranged in series.

In order to ease the detection of viral particles existing in a biological sample, said device could further include an antechamber configured to convey said biological sample to be analysed at said biosensors.

In a preferred embodiment according to the invention, said antechamber could be implemented in tubular or cylindrical shape, and could include an interchangeable disposable end, suitable for the analysis of a sample of exhaled of a subject. By exhalation by a subject through the disposable end of the tubular antechamber of the device, possible viral particles existing within the subject's exhaled could be transported effectively at the active area of the biosensor. By pure way of example, a graphic representation of an embodiment of the device for analysing a sample of exhaled of a subject is shown in FIG. 10.

The device antechamber alternatively could be configured so as to receive a liquid sample to be analysed, for example a sample of serum, saliva or any other liquid sample suitable for the detection of viral particles, and to vehiculate it at the active surface of the sensor.

According to an aspect of the present invention, said device could further include means for processing an electric signal produced by said one or more biosensors, configured to detect said electric signal and to process it in output data comprising information about the presence of viral particles in said biological sample. Not limiting examples of means for processing said electric signal comprise amplifying systems, filter, electric meters, multipliers and/or any other processing means known in the art.

According to an aspect of the present invention, said processing means comprises at least a multiplexer and/or one or more electric meters. Said multiplier can be used specifically to select single FETs (or gFETs) for the application of appropriate polarization voltages, and for transmitting the electric signal generated to one or more high-resolution electric meters capable of measuring small electric variations emitted by the transistors in response to the virus detection.

Said device could further include a microcontroller which can supervise the procedures of the whole system and launch the detection algorithms.

Said device could also include means for transmitting said information associated to said processing means. Means for transmitting said information will include at least one between displaying unit of said output data, for example a screen, and/or radio frequency transmission unit, for example a unit for transmitting by Wi-Fi, Bluetooth or infrared; and/or serial transfer unit, for example a USB port.

According to an aspect of the present invention, said device will include a power supply system. In an embodiment, said device could include, in particular, a series of logic components as schematized in the diagram in FIG. 9. A preferred embodiment according to the present invention in particular relates to a portable device comprising supporting means apt to house at least a field-effect transistor biosensor according to any one of the herein described embodiments, and signal acquisition means operatively connected thereto.

Supporting means suitable to be used for implementing a device according to the present invention is exemplified in FIGS. 14B and 14C, and comprises a plurality of casings and at least a housing slot for a biosensor according to any one of the previously described embodiments.

Signal acquisition means suitable to be used in a device according to the present invention comprises processing means, transmission means, a microcontroller and a power supply system according to any one of the herein described embodiments.

Said signal acquisition means in particular comprises means capable of detecting the shifting of the dirac point in I-V curve after a specific interaction occurred between the protein immobilized on the biosensor surface according to any one of the herein described embodiments and a viral particle and/or a fragment thereof present in a sample subjected to analysis.

Preferably, said signal acquisition means will include a series of logic components as schematized in FIGS. 13A and 13B.

According to an aspect of the invention, the signal acquisition means could be housed within a supporting element comprising at least an upper casing and a lower casing, preferably comprising at least a window and a switch-on button, as for example shown in FIGS. 14A and C.

As previously mentioned, the generation of a detectable electric signal in response to the recognition of a viral particle and/or a fragment thereof by a protein suitably oriented and immobilized on the surface of the substrate of a FET biosensor according to any one of the herein described variants, offers the advantage of being able to detect quickly, with high selectivity and sensitivity, the presence of said viral particles in a biological sample.

Therefore, the present invention also relates to the in vitro use of a biosensor or a device according to any one of the herein described embodiments for the diagnosis of viral infections. According to a preferred embodiment of the present invention, said viral infections in particular are SARS-COV-2 infections.

Advantageously, the capability of the biosensor of the present invention of detecting whole viral particles can be used also for the detection of viral particles existing in the environment; therefore, the present invention also relates to the use of a biosensor or a device according to any one of the previously described embodiments for the detection of viral particles in environmental matrices, such as for example air or water.

An additional aspect of the invention relates to un method for detecting the presence of a viral particle and/or a fragment thereof within a biological sample and/or for the diagnosis of a viral infection comprising the following steps:
i. contacting a biological sample to be analysed with a biosensor or with a device comprising one or more biosensors according to any one of the previously described embodiments; and
ii. detecting binding reactions with a viral particle and/or a fragment thereof by means of an electrical signal emitted by said one or more biosensors.

A biological sample to be analysed according to the present invention can be a sample of saliva, blood, urine, mucus, nasopharynx and/or pharyngeal mucosa, exudate, sputum, and/or exhaled of a subject.

The sample to be subjected to the analysis by means of a biosensor or a device according to any one of the herein described embodiments can be a sample obtained from an asymptomatic subject, or from a subject who already has symptoms of viral infection, such as for example, in case of SARS-COV-2 infection, symptoms of moderate entity, such as fever, cough, shortness of breath, gastrointestinal disorders, or more severe symptoms, such as pneumonia.

According to an aspect of the present invention, the herein described method can include an additional step:
iii. diagnosing said viral infection when said biosensor and/or device produces a detectable electrical signal in response to binding with said viral particle and/or a fragment thereof.

As previously mentioned, the production of an electric signal derives from a variation in the charge mobility of the substrate induced by the reaction of biological binding, which is capable of determining a change in the electric conductivity of the substrate itself.

In an embodiment according to the present invention, said viral particle is a particle of SARS-CoV-2 and/or a fragment thereof.

The present invention also relates to a method for detecting the presence of a viral particle and/or a fragment thereof within an environmental matrix comprising the following steps:
i. contacting a sample collected from an environmental matrix to be analysed with a biosensor or with a device comprising one or more biosensors according to any one of the previously described embodiments; and
ii. detecting binding reactions with a viral particle and/or a fragment thereof by means of an electrical signal emitted by said one or more biosensors.

According to an aspect of the present invention, samples collected from environmental matrices which can be analysed by means of a biosensor or a device according to the herein described method preferably include samples of air and/or water.

The present invention also relates to a process for the preparation of a field-effect transistor (FET) biosensor for the detection of viral particles and/or fragments thereof comprising the following steps:
i. arranging a FET biosensor comprising at least a substrate;
ii. functionalizing said at least a substrate with a linker molecule capable of binding a first single-stranded oligonucleotide;
iii. binding a protein capable of specifically binding a viral particle and/or a fragment thereof to a second single-stranded oligonucleotide comprising a nucleotide sequence complementary to the nucleotide sequence of said first oligonucleotide;
iv. immobilizing said protein bound to said second oligonucleotide on the surface of said substrate by hybridization of said first oligonucleotide with said second oligonucleotide.

According to an aspect of the present invention said FET biosensor comprises at least a substrate made of graphene. In an embodiment of the invention, said FET biosensor comprises at least a graphene-based FET device as described in the data sheet of the gFET-S20 (Graphenea) product, herein incorporated as reference.

According to an aspect of the present invention, said process can include, after step (ii) and before step (iv), one or more steps for washing said substrate with an organic solvent and/or with deionized water so as to remove the excess of linker molecules not immobilized on the substrate surface, and/or at least a step of treatment with a solution comprising a compound selected among glycine, amino-polyethylene glycol 5-alcohol (PEG) and/or ethanolamine, so as to ending the groups of the linker molecule which have not been conjugated, or to cover the substrate portions left uncovered and then to maximize the interaction specificity.

In an aspect of the present invention, the step (iii) of the herein described process can be carried out by means of any one of the reactions known in the art for the formation of protein-DNA hybrid complexes. According to a preferred embodiment, said step (iii) can be carried out by means of a reaction mediated by Sortase enzyme. Preferably, said first oligonucleotide is bound to the C-terminal portion of said protein.

The step (iv) of the herein described process can be carried out at room temperature, for an overall period of time comprised between 1 to 4 hours.

In an embodiment according to the invention, said at least a substrate is graphene, said linker molecule is 1-pyrenebutyric acid N-hydroxy succinimide ester, said protein is ACE2 viral receptor, or an engineered version thereof, said first oligonucleotide has the sequence 5′-GCACTG-3′ and said second oligonucleotide has the sequence 5′-CAGTGC-3′, wherein said second oligonucleotide comprises an amino group at the 5′-end, and wherein said ACE2 receptor is bound to said second oligonucleotide through the C-terminal portion.

The present invention also relates to a FET biosensor functionalized according to any one of the previously described embodiments, which can be obtained by means of said process.

An aspect of the present invention also relates to a kit for the detection of a viral particle and/or a fragment thereof and/or for the diagnosis of a viral infection comprising a biosensor and/or a device according to any one of the previously described embodiments, and one or more control reagents.

Control agents suitable to be used in a kit according to the present invention include negative control reagents, for example solutions comprising proteins, such as viral proteins, which are not capable of specifically binding said viral receptor immobilized on the substrate surface of said biosensor. In an embodiment according to the present invention, said kit comprises a solution of SARS-COV-2 mPRO protease as negative control reagent.

Examples are reported herebelow having the purpose of better illustrating the compositions and methods detected in the present description, such examples are not to be considered in any way as a limitation of the preceding description and of the subsequent claims.

### Examples

### Example 1—Analysis of Graphene-Based FET Electric Behaviour

In case of a gate based upon an ionic liquid, particularly useful for the biosensing and biological applications, the double layer at the graphene-ionic liquid interface carries out the role of insulating spacer between gate and channel, and it is associated to a geometric capacity. The quantum capacity of the single-layered graphene provides an additional contribution which, in an ionic liquid electrolyte, is typically comparable or dominant with respect to the geometric capacity and depends upon the gate voltage through the density of surface charge induced on the graphene. The chemically inert liquid ionic gate is characterized by a length of Debye ionic shielding of few Angstroms, depending upon the different ionic concentrations of the aqueous solutions. The typical values measured for the geometric capacity CG are of the order of few tens of μF/cm².

The geometric capacity has the following expression:

$$\sqrt{{C}_{Q} = \frac{{e}^{2}}{π ℏ {v}_{F}} \sqrt{n}}$$

wherein v_{F} represents Fermi velocity in graphene, “e” is the unitary charge, “n” is the surface charge density of graphene (for the lacunae there is a mirror analysis), depending upon V_{G}. From a theoretical point of view, a rigorous analysis of the potential (Poisson equation) and of the charge density n (Schroedinger or Dirac equations, and diffusion effects) are required so that a self-consistent characterization of the device could be performed. The two different contributions of the capacities can be modelled as in-series parallel plate capacitors:

$$\sqrt{n = 1 / ( \frac{1}{{C}_{Q}} + \frac{1}{{C}_{G}} ) {V}_{G}}$$

In this formula C_{G} depends upon n, or, in equivalent way, upon V_{G} through the level of Fermi E_{f} of graphene. Generally, the electric current can be expressed, by neglecting the diffusion effects, through the electron drift and lacunae under the external potential V_{D},

$$I = \sqrt{\frac{W}{L} ( \vert e \vert μ n + \vert e \vert μ p ) {V}_{D}}$$

wherein p is the lacuna density.

The charge density in the real graphene is influenced by the presence of various impurities and defects, due for example to the substrate, which play an important role in the properties of transportation near Dirac point: the fluctuations in the relative potential, in a self-consistent description, provide that n includes an additional contribution of density of charge carriers, n*, which can be set to a value different from zero by varying V_{G}, so as to obtain a current minimum (value not equal to zero) in curves I-V_{G}. In the detail, the charge density on graphene is generally influenced by residual impurities, whereas the electronic structure is approximately unchanged. Moreover, two doping mechanisms for transferring the charge on the graphene surface by means of external perturbation are possible: electronic doping and electrochemical doping. The electronic doping provides a direct charge transfer between the graphene and an adsorbed compound. The chemical doping provides the participation of the adsorbed substance in electrochemical redox reactions in which the graphene acts as electrode.

When the graphene in the liquid gate is perturbated by different charged species, the curve I-V_{G} is affected both in terms of residual charge density and position of Dirac point. Generally, the effect of gate voltage V_{G} can be described as follows: the charge related to the electrostatic (or electronic) doping is modified with V_{G} according to the self-consistent charge potential analysis or, approximately, according to the simple equivalent circuit model given by two in-series capacities as mentioned above. Therefore, the effect of a perturbation external to the charge density (doping) given by the biomolecular interaction is double and it involves:
a lateral displacement of the minimum of I-V_{G} curve (position of Dirac point) in case of electronic doping.
a vertical variation of the minimum of I-V curve upon varying the density of residual charge n*.

At last, the variation in I-V_{G} feature, due to a variation in drain voltage VD, is given, ideally and according to theory, by a simple displacement of Dirac point, approximatively by an amount equal to V_{D}/2.

### Example 2—Simulations of Molecular Dynamics

The binding between ACE2 receptor and the receptor-binding-domain (RBD) was well characterized by means of several structural studies. Starting from these pieces of information, the dynamic behaviour at the interface was studied by means of simulations of molecular dynamics, by obtaining a complete dynamic description of the ACE2-bound state. Several repetitions of simulations of molecular dynamics were also performed, oriented so as to characterize the detachment of a receptor-binding-domain (RBD) of S (Spike) protein from human ACE2 receptor. These simulations allowed to highlight the main interactions at the interface which were used to define the ACE2 region. The force requested to separate a monomer of S protein from the receptor was quantified, by performing simulations of molecular dynamics oriented by 275 ns in which the traction speed was kept constant at a value equal to 0.05 Å/ns, obtaining a value around 500 pN (FIG. 5) sufficient to keep the binding.

### Example 3—Functionalization of the Graphene Substrate

Several experiments were performed by using a not-functionalized graphene-based FET (gFET) or graphene-based FET biosensors respectively functionalized with a selected portion of the human ACE2 receptor, with ACE2 functionalized with His-Tag at the C-terminal portion, or with Fc-ACE2 Chimera and with the nanobodies functionalized with His-Tag at the N-terminal portion.

ACE2-His-Tag, Fc-ACE2, Nbs-His-Tag receptors were respectively immobilized covalently on the corresponding devices by means of the PBASE linker molecule. A drop of PBASE 2-5 mM (Thermo Fisher Scientific, Waltham, MA) made of dimethylformamide (DMF) was positioned on graphene-based (gFET-S20, Graphenea) chip for 2 hours at room temperature. Each chip was then subjected to several washing with DMF, deionized water (DI) to remove the not bound reaction excess and it was dried by means of N2. Each device functionalized with PBASE was then subjected to 250 μg/mL of ACE2-His-Tag (10108-H08B-100; Sino Biological, Inc., China) (or, Fc-ACE2 (Z03484,GenScript) or Nbs-His-Tag respectively) receptor and left for one night in environment humidified at 4° C., in an aqueous solution of PBS 1X. The sensors were subsequently washed sequentially in PBS (pH 7.4, 1X) and in deionized water so as to remove the whole not bound protein excess and dried under nitrogen flow.

The chips were subsequently treated with a solution selected among glycine, amino-polyethylene glycol 5-alcool (PEG) and ethanolamine, in particular with glycine 100 mM in PBS (pH 7.4, 1X) for 30 minutes, at room temperature, for the termination of NHS groups of PBASE which were not conjugated, or to cover the graphene portions remained uncovered and then to maximize the interaction specificity. After the treatment with glycine, the samples were washed with PBS (pH 7.4, 1X), deionized water and dried with N₂.

In order to confirm the occurred functionalization on the graphene surface by using PBASE and ACE2, images of atomic force microscopy (AFM) of the not functionalized graphene (FIG. 6a) and of the graphene modified with PBASE (FIG. 6b) were obtained, for which in particular heights of peaks equal to about ˜2 nm were detected, by suggesting the presence of the linker. At last, the graphene functionalized with ACE2 incubated in presence of S1 subunit of SARS-COV-2 Spike protein (FIG. 6c) was analysed. The length of the detected peak is comprised between ˜15 and ˜20 nm, by confirming the presence of PBASE and both proteins (that is ˜2 nm PBASE+˜10 nm ACE2+˜8 nm domain S1).

### Example 4—Immobilization of ACE2 on Graphene by Hybridization of Two DNA Probes

ACE2 receptor was immobilized covalently on the device by hybridization of two DNA probes: 5′-GCACTG-3′ and H2N-5′-CAGTGC-3′.

A drop of PBASE 2-5 mM (Thermo Fisher Scientific, Waltham, MA) made of dimethylformamide (DMF) was positioned on graphene-based (gFET-S20, Graphenea) chip for 2 hours at room temperature. Each chip was then subjected to several washing with DMF and deionized water (DI) to remove the excess of not bound reagent, and it was dried by means of N2. The device functionalized with PBASE was then exposed to an aqueous solution of DNA H2N-5′-CAGTGC-3′ probe and incubated for one night in environment humidified at 4° C. or for 3 hours at room temperature. Not bound DNA excess was eliminated through repeated washing with PBS (pH 7.4, 1X) and deionized water.

The chip was then subsequently treated with a solution selected among glycine, amino-polyethylene glycol 5-alcohol (PEG) and ethanolamine, for example with glycine 100 mM in PBS (pH 7.4, 1X) for 30 minutes, at room temperature, for the termination of the NHS groups of PBASE which were not conjugated, or to cover the graphene portions left uncovered and then to maximize the interaction specificity. After the treatment with glycine, the samples were washed with PBS (pH 7.4, 1X), deionized water and dried with N2.

At this point a solution containing ACE2 protein modified at C-terminal portion, by means of Sortase technology, with an oligonucleotide complementary to the one already immobilized on the substrate by means of PBASE (ACE2 C-term-5′-GCACTG-3′), was added to the chip. In order to obtain hybridization of the two DNA probes, the sample was incubated for several hours (1-4 hours) at room temperature. Subsequently, the sample was washed with PBS 1X and deionized water.

### Example 5—I-V Curve

The current/resistance-voltage curves for the graphene-based FET in liquid gate by means of PBS (Phosphate saline buffer at pH 7.4) were acquired by using the electrode on AuPd chip.

A series of gFET under different conditions of gating in liquid was characterized by using a Wentworth probe station with Bausch & Lomb MicroZoom microscope and a HP4145B analyser of semiconductor parameters. The drain-source currents were measured in function of the drain-source voltages for different gate-source polarizations. In particular, for each gFET: i) the channel resistance was measured as a function of the voltage of liquid gate for different drain-source voltages; ii) the drain-source current was measured as a function of the drain-source voltage for several voltages of liquid gate. As example, the channel resistance for four drain-source voltages (10, 50, 90 and 130 mV) as a function of the gate-source voltage, comprised between 0 and 1.5 V, is shown in FIG. 8. The resistance is shown in both graphs both for the functionalization of liquid gate with ACE2 receptor (blue line) and for the functionalization with SARS-COV-2 Spike protein (S protein) (red line), respectively. It is possible to appreciate a clear variation (increase) in the gate voltage. This confirms the possibility of using this parameter to detect the presence of Spike S1 protein.

Also the results are discussed hereinafter obtained by functionalizing the gFET with ACE2 protein with His-tag (FIG. 11) and with Fc-ACE2 (Fc-tag) (FIG. 12) and by using as analysis samples two proteins produced by SARS-COV-2 virus: Spike protein in trimeric form and a protein called mPRO, a protease produced by the virus which is not recognized by the ACE2 receptor and then acts as appropriate negative control for the interaction specificity.

As it is shown in the graphs A, B and C in FIGS. 11 and 12, the occurred interaction between the receptor and Spike protein is demonstrated by a clear variation in the gate potential (Vg), which is recognized as indicator of electronic perturbation on the biosensor surface indeed given by the binding of Spike with the receptor adherent on the graphene. The same variation instead is not detected after the addition of control mPRO protein (graphs D, E, F), by confirming the system specificity in recognizing Spike protein. Both ACE2 versions functionalized on the graphene surface showed a detectable signal, although that with tag Fc (FIG. 12) produced a wider signal the analyte concentration being equal, by improving the system sensitivity.

### Example 6—Device Prototyping

The biosensor (meant as chip functionalized with ACE2 receptor-Fc) according to the present invention was integrated within a device consisting of a support (that is “carrier”) for the chip and a casing (that is “case”) consisting of electronic portions. The Point-Of-Care (POC) device is a battery-powered device for the high-precision signal acquisition, custom designed and connected to a casing (that is, case) containing a replaceable gFET biosensor (FIG. 13B). This communicates with the measurement system on PC through a wireless connection of Bluetooth Low Energy (BLE) type.

The measurement electronics include a low dispersion input multiplexer (MUX) to allow the automatic scanning of all 12 transistors on the graphene chip, high-resolution 16-bit DAC to set the potentials of gate and drain, buffer to drive the gFET transistors and a high-resolution 31-bit ultra-ADC to detect the drain current (FIG. 13A).

The biosensor casing (that is, case) is devised to be economic and easily replaceable, it does not contain active current and provides only electrical connections to the gFET terminals, which are connected to the main device through a 24-pole USB-C connector 24 (FIG. 13B).

DAC (digital-to-analog converter) has a resolution of about 20 uV, the system was created to have performances comparable to the instrument used in the previous preliminary measurements but in a portable and miniaturized device.

### Drawing of Case and Carrier Unit

The signal acquisition device consists of four main portions: an upper casing, a lower casing, a window and a switching-on/off button (FIG. 14A). The final prototype was printed in 3D by using the SLS (Selective Laser Sintering) printing technique. Near the main casing (FIG. 14C), the casing for gFET (FIG. 14D) was designed. This unit keeps together the graphene chip and the spring contact pins, by guaranteeing a stable and precise alignment therebetween. The casing for the gFET consists of three main portions: Carrier 1, Carrier 2 and Carrier 3 (FIG. 14B). Carrier 1 houses the casing in which the spring-loaded contact pins are incapsulated. Carrier 2 has a slot including the 10,4×10,4-mm gFET chip. Carrier 2 also has housings with hexagonal shape in which it is possible to insert M3 nuts. Carrier 3 acts as base which keeps together the whole group.

### Experimental Data (Isolated Virus and Patients Positive to SARS-COV-2)

The device was then tested experimentally by using different SARS-COV-2 depowered variants (under depowered virus an isolated virus, rendered biologically inactive, is meant, but capable of maintaining the antigenic properties), apart from samples coming from swabs of patients positive to SARS-COV-2. For each set of experiments also the corresponding negative controls were analysed.

Moreover, in order to test the specificity, our biosensor was tested with a Spike sample coming from MERS virus, progenitor of the current SARS-COV-2 virus, uncapable of recognizing ACE2 receptor (used in our system as bioreceptor). The experimental test on MERS spike did not involve any significative signal by confirming the absolute biosensor specificity (FIG. 15).

Under the expression “signal” the displacement of dirac point in the I-V curve after a specific interaction, occurred between the bioreceptor functionalized on graphene and a molecule existing in solution, is meant. In FIGS. 15-17 there are graphs representing the dirac point normalized before (in black) and after (in grey) incubation with a sample.

Once tested the system specificity, the prototype was used to analyse currently existing depowered viruses of different SARS-COV-2 variants, in order to evaluate the capability thereof in recognizing each one thereof (FIG. 16). The negative control is represented by the isolated Human Herpes Virus (HSV-1).

As shown by FIG. 16, the biosensor according to the present invention, differently from many antigenic tests currently existing on the market, is capable of recognizing each currently known virus variant. The lack of an appreciable signal in HSV-1 sample confirms once again the system specificity.

The tested SARS-COV-2 virus variants were:
D614G (two different samples)
Alpha
Beta
I Gamma
I Delta (two different samples).

By considering the promising results obtained by using depowered viruses, the subsequent step was to test the biosensor of the invention with samples of real patients. In order to perform the experiment nasopharyngeal swabs kept in physiological solution (PBS 1x) of eight different patients were used which, after PCR molecular swab and subsequent characterization, resulted to be: two negative, six positive for the following variants: D614G, Alpha, Gamma, Delta (two patients), Omicron. The obtained results (FIG. 17) demonstrate the detection of the different viral variants, including Omicron, by the biosensor the present invention relates to. The two negative samples did not show a significantly appreciable signal.

LIST OF SEQUENCES IN THE DESCRIPTION

SEQ ID Nr: 1 Aminoacidic sequence of ACE2 receptor 1-732 (NCBI Reference Sequence:
NP_001358344.1)
MSSSSWLLLSLVAVTAAQSTIEEQAKTFLDKENHEAEDLFYQSSLASWNYNTNITEE
NVQNMNAGDKWSAFLKEQSTLAQMYPLQEIQNLTVKLQLQALQQNGSSVLSEDKSKR
LNTILNTMSTISTGKVCNPDNPQECLLLEPGLNEIMANSLDYNERLWAWESWRSEVG
KQLRPLYEEYVVLKNMARANHYEDYGDYWRGDYEVNGVDGYDYSRGQLIEDVEHTFE
EIKPLYEHLHAYVRAKLMNYPSYISPIGCLPAHLLGDMWGREWTNLYSLTVPFGQKP
NIDVTDAMVDQAWDAQRIFKEAEFFVSVGLPNMTQGFWENSMLTDPGNVQKAVCHPT
AWDLGKGDFRILMCTKVTMDDELTAHEMGHIQYDMAYAAQPELLRNGANEGFHEAVG
EIMSLSAATPKHLKSIGLLSPDFQEDNETENFLLKQALTIVGTLPFTYMLEKWRWMV
FKGEIPKDQWMKKWWEMKREIVGVVEPVPHDEYCDPASLFHVSNDYSFIRYYTRTLY
QFQFQEALCQAAKHEGPLHKCDISNSTEAGQKLENMLRGKSEPWTLALENVVGAKNM
NVRPLLNYFEPLFTWLKDQNKNSFVGWSTDWSPYADQSIKVRSLKSALGDKAYEWND
NEMYLERSSVAYAMRQYFLKVKNQMILFGEEDVRVANLKPRISENEFTAPKNVSDII
PRTEVEKAIRMSRSRINDAFRLNDNSLEFLGIQPTLG
Nucleotide sequence of the first oligonucleotide
GCACTG
Nucleotide sequence of the second oligonucleotide
CAGTGC
SEQ ID No: 2 Aminoacidic sequence of the motif recognized by sortase enzyme
LPXTG
SEQ ID No: 3 Aminoacidic sequence of the nanobody Ty1 (Hanke L. et al. “An alpaca nanobody
neutralizes SARS-CoV-2 by blocking receptor interaction” Nat. Comm. 11, N. 4420 (2020))
QVQLVETGGGLVQPGGSLRLSCAASGFTFSSVYMNWVRQAPGKGPEWVSRISPNSGNIGYTDS
VKGRFTISRDNAKNTLYLQMNNLKPEDTALYYCAIGLNLSSSSVRGQGTQVTVSS
SEQ ID No: 4 Aminoacidic sequence of the nanobody His-tagged H11-H4_(Huo J. et al.
“Neutralizing nanobodies bind SARS-CoV-2 spike RBD and block interaction with ACE2” Nat.
Structural & Molec. Biol., 27, 846-854 (2020))
QVQLVESGGGLMQAGGSLRLSCAVSGRTFSTAAMGWFRQAPGKEREFVAAIRWSGGSAYYADS
VKGRFTISRDKAKNTVYLQMNSLKYEDTAVYYCAQTHYVSYLLSDYATWPYDYWGQGTQVTVSS
KHHHHHH
SEQ ID No: 5 Aminoacidic sequence of the nanobody His-tagged H11-D4_(Huo J. et al.
“Neutralizing nanobodies bind SARS-CoV-2 spike RBD and block interaction with ACE2” Nat.
Structural & Molec. Biol., 27, 846-854 (2020))
QVQLVESGGGLMQAGGSLRLSCAVSGRTFSTAAMGWFRQAPGKEREFVAAIRWSGGSAYYADS
VKGRFTISRDKAKNTVYLQMNSLKYE
DTAVYYCARTENVRSLLSDYATWPYDYWGQGTQVTVSSKHHHHHH

1. A field-effect transistor (FET) biosensor for the detection of viral particles and/or fragments thereof comprising a substrate and a protein capable of specifically binding a viral particle and/or a fragment thereof, immobilized on the surface of said substrate, wherein said protein is immobilized so that the binding of a viral particle and/or of a fragment thereof to said protein determines the emission of a detectable electrical signal.

2. The biosensor according to claim 1, wherein said protein is a protein capable of specifically binding a viral particle of SARS-COV-2 and/or a fragment thereof.

3. The biosensor according to claim 1, wherein said substrate is graphene.

4. The biosensor according to claim 1, wherein said protein is immobilized on the surface of said substrate through the binding of a group of said protein with a linker molecule conjugated to the surface of said substrate.

5. The biosensor according to claim 4, wherein said group of said protein is the C-terminal portion of the aminoacidic sequence of said protein.

6. The biosensor according to claim 1, wherein said protein is immobilized on the surface of said substrate by hybridization of a first single-stranded oligonucleotide bound to said protein with a second single-stranded oligonucleotide bound to a linker molecule conjugated to the surface of said substrate.

7. The biosensor according to claim 6, wherein the 5′-end of said first oligonucleotide is bound to the C-terminal portion of the aminoacidic sequence of said protein.

8. The biosensor according to claim 6, wherein the 5′-end of said first oligonucleotide is bound to the C-terminal portion of said protein by means of a conjugation reaction mediated by the sortase enzyme.

9. The biosensor according to claim 6, wherein said second oligonucleotide comprises an amino group at the 5′-end capable of binding said linker molecule conjugated to the surface of said substrate.

10. The biosensor according to claim 6, wherein said first and said second oligonucleotide consist of a number of bases comprised between 6 and 8.

11. The biosensor according to claim 6, wherein said first oligonucleotide has the sequence 5′-GCACTG-3′ and said second oligonucleotide has the sequence 5′-CAGTGC-3′, wherein said second oligonucleotide comprises an amino group at the 5′-end.

12. The biosensor according to claim 4, wherein said linker molecule is 1-pyrenebutyric acid N-hydroxy succinimide ester.

13. The biosensor according to claim 1, wherein said protein is capable of binding the S1 subunit of the SARS-COV-2 Spike protein.

14. The biosensor according to claim 1, wherein said biosensor is a graphene-based chip having dimensions equal to 10 mm×10 mm.

15. The biosensor according to claim 1, wherein said graphene-based chip consists of 12 graphene-based field-effect transistors (GFET).

16. The biosensor according to claim 1, wherein said protein is a viral receptor or a nanobody.

17. The biosensor according to claim 1, wherein said protein is the angiotensin-converting enzyme 2 (ACE2).

18. The biosensor according to claim 1, wherein said protein is the ACE2 enzyme having sequence SEQ ID Nr. 1, wherein one or more of the following mutations are inserted in the sequence of said enzyme: T27Y, L79T, N330Y.

19. A portable device comprising field-effect transistor biosensor according to claim 1.

20. The device according to claim 19, comprising a plurality of said biosensors arranged in series.

21. The device according to claim 19, further comprising an antechamber configured to convey a biological sample to be analysed at said biosensors.

22. The device according to claim 19, further comprising means for processing an electric signal produced by said one or more biosensors, configured to detect said electric signal and to process it in output data comprising information about the presence of viral particles in said biological sample.

23. The device according to claim 22, wherein said processing means comprises at least a multiplexer and/or one or more filters.

24. The device according to claim 23, further, comprising means for transmitting said information associated to said processing means.

25. The device according to claim 24, wherein said transmission means comprises at least one among:
displaying unit of said output data, and/or
radio frequency transmission unit; and/or
serial transfer unit.

26. (canceled)

27. (canceled)

28. A method for detecting the presence of a viral particle and/or a fragment thereof within a biological sample and/or for the diagnosis of a viral infection comprising the following steps:
i. contacting a biological sample to be analysed with a biosensor of claim 1; and
ii. detecting binding reactions with a viral particle and/or a fragment thereof by means of an electrical signal emitted by said biosensor.

29. The method according to claim 28, wherein said biological sample to be analysed is a sample selected from the group consisting of saliva, blood, urine, mucus, nasopharynx and/or pharyngeal mucosa, exudate, sputum, and/or exhaled of a subject.

30. The method according to claim 28, further comprising the following step:
iii. diagnosing said viral infection when said biosensor and/or device produces a detectable electrical signal in response to binding with said viral particle and/or a fragment thereof.

31. The method according to claim 28, wherein the biosensor protein binds to the S1 subunit of the SARS-COV-2 Spike protein.

32. A process for the preparation of a field-effect transistor (FET) biosensor for the detection of viral particles and/or fragments thereof comprising the following steps:
i. Arranging a FET biosensor comprising a substrate;
ii. Functionalizing said substrate with a linker molecule capable of binding a first single-stranded oligonucleotide;
iii. binding a protein capable of specifically binding a viral particle and/or a fragment thereof to a second single-stranded oligonucleotide comprising a nucleotide sequence complementary to the nucleotide sequence of said first oligonucleotide; and
iv. conjugating said protein bound to said second oligonucleotide to said substrate, by hybridization of said first oligonucleotide with said second oligonucleotide.

33. The kit for the detection of a viral particle and/or a fragment thereof and/or for the diagnosis of a viral infection comprising a biosensor according to claim 1, and one or more control reagents.
